(19) United States
(12) Patent Application Publication
(10) Pub. No.: US 2018/0301218 A1
(43) Pub. Date: Oct. 18, 2018

(54) SMART HEALTHCARE STAFFING SYSTEM FOR HOSPITALS

(71) Applicant: PRAOS HEALTH INC., AUSTIN, TX (US)

(72) Inventor: Rohini Rao BOCHATON, Austin, TX (US)

(21) Appl. No.: 15/902,777

(22) Filed: Feb. 22, 2018

Related U.S. Application Data
(60) Provisional application No. 62/484,973, filed on Apr. 13, 2017.

(51) Int. Cl. G16H 40/20; G06Q 10/10

(52) U.S. Cl. CPC G16H 40/20; G06Q 10/1053

(57) ABSTRACT

A decision support tool to assist decision-making in the field of temporary staffing for any type of short term specialist hiring where time and skill match are of the essence. The system includes networked, standalone, programmed devices that contain several integrated components. One component is a data storage subsystem that stores information about an employer and a candidate. A data entry subsystem component allows input of hiring requirements of the employer and of credentials of the candidate. A data analytics component compares the hiring requirements with the credentials and produces a match. A workflow management component manages a professional engagement resulting from the produced match. The data analytics component that produces the match and the workflow management component that manages the professional engagement are integrated in a networked computerized system.

This application claims priority to Provisional application No. 62/484,973, filed on Apr. 13, 2017.

### TECHNICAL FIELD

The presently disclosed subject matter relates to a staffing and recruitment fulfillment system for healthcare providers. In particular, the presently disclosed subject matter relates to a transparent and timely placement system for nurses, designed to maximize flexibility, optimize personnel costs and improve patient satisfaction.

### BACKGROUND

Conventional systems for managing staffing demand and supply between healthcare institutions and professionals offer solutions that are incomplete at best, and outdated and inefficient in most cases. Needless to say, keeping a sufficient number of permanent staff, such as nurses for example, to cover every feasible exigency is an unacceptable expense and is economically unfeasible. Thus, hospitals resort to using float pools, where an individual nurse is selected from a pool when needed. In this system, the governing criterion for selection is availability, which is largely a manual process, whereby hospital staff contacts the nurse pool via telephone and email to establish their availability. Very often there is rate negotiation to entice them to fill a shift. If the position goes unfilled due to time delays with float pool responses or the negotiation process, hospitals may have to divert patients to another facility due to inadequate staff coverage, due to their obligation to maintain minimum nurse to patient ratios. The diversions are detrimental to patient satisfaction.

Another conventional option has been using staffing agencies for locating available nurses. Hospitals reach out to staffing agencies in an attempt to improve flexibility between the periods of high and low demand at a hospital by seeking contingent labor. Nonetheless, the process that staffing agencies apply to match a hospital with a nurse is largely manual (using phone, paper, fax) and inefficient. Typically, this process entails searching for a nurse, establishing rapport between the hospital and the nurse, and performing related steps, such as vetting the nurse's credentials, each time help is needed. Moreover, the quality of the actual relationship between the hospital and the nurse and the fulfillment of the required duties is normally not monitored or managed by staffing agencies. Such additional steps, including even the basic “handshake” establishment by a staffing agency, amount to a considerable expense to the hospital. The “middleman” cost affects the hospital's competitiveness in terms of compensation, or, in the alternative, the added cost decreases profitability of the hospital thereby diminishing its ability to invest in providing the best possible healthcare services to its patients.

Recently, solutions to eliminate the middle-man and automate the ‘candidate matching’ such as a web-based online marketplace connecting doctors with freelance clinical work have emerged. These type of platforms have not been mobile-enabled and have not been designed for just-in-time staffing. Instead, they have been akin to job boards. While variations of an online marketplace have been envisioned as replacement for the staffing agencies, none of these solutions to date automate the current real life candidate engagement, selection, monitoring, compliance checking, and job supervision process in near real time, which is a requirement for just-in-time staffing.

In light of the discussed inadequacies of the existing technology, there is a need for a tool that is capable of providing an optimal match between a hospital and a nurse in a timely manner, while integrating the process of recruitment with the professional relationship between the employer and the employee, from the moment of demand until the moment when the nurse is released from duty and compensated.

### SUMMARY

The presently disclosed subject matter relates to decision making tool, which is a system used for integrating and processing staffing information, or for any type of short term specialist hiring where time and skill match are of the essence. Within the healthcare domain, short term specialists may include nurses, occupational therapists, physical therapists, speech therapists, radiologists, and physician assistants, among others. Other highly specialized contractors using the system may be Government cleared security experts, oil and natural gas experts with special credentials, and professionals for whose selection time and skill match is a key requirement. The staffing information may contain at least one of the following: healthcare, education, security, drilling, disaster recovery and emergency information

The system includes a plurality of networked, standalone, programmed devices, and a network that connects the networked, standalone, programmed devices. In one embodiment, each of the plurality of networked, standalone, programmed devices includes: a data storage subsystem component that stores information about at least one employer and at least one candidate, a data entry subsystem component that allows input of hiring requirements of the at least one employer and of credentials of the at least one candidate, a data analytics component programmed to compare the hiring requirements with the credentials and to produce a match between the hiring requirements and the credentials, and a workflow management component programmed to manage a professional engagement resulting from the produced match. In one example, the data analytics component that produces the match and the workflow management component that manages the professional engagement are integrated in the networked computerized system.

The workflow management component may be used for negotiating terms of employment of the at least one candidate. Further, the workflow management component may be used for compensation of the at least one candidate and/or for performance rating of at least one of the following: the at least one candidate and the at least one employer. The workflow management component may also be used for supervision by the at least one employer of job performance of the at least one candidate. The workflow management component may provide performance review data to the data storage subsystem component.

In another embodiment, a method for integrating and processing staffing information, comprises: storing information about at least one employer and at least one staffing candidate by using a data storage subsystem component of a networked computerized system, inputting hiring requirements of the at least one employer and of credentials of the at least one candidate by using a data entry subsystem component of the networked computerized system, comparing the hiring requirements with the credentials and producing a match between the hiring requirements and the credentials by using a data analytics component of the networked computerized system, and managing a professional engagement resulting from the produced match by a workflow management component of the networked computerized system. The data analytics component that produces the match and the workflow management component that manages the professional engagement may be integrated in the networked computerized system.

### BRIEF DESCRIPTION OF THE DRAWINGS

FIG. 1 shows an example of a field user work flow of the smart healthcare staffing system.

FIG. 2 shows an example of an employer work flow of the smart healthcare staffing system.

FIG. 3 shows an example of a mobile signup for a field user mobile application of the smart healthcare staffing system.

FIG. 4 shows an example of a field user account creation for the smart healthcare staffing system.

FIG. 5 shows an example of a field user portal for the smart healthcare staffing system.

FIG. 6 shows a block diagram illustrating a computing system for implementing the smart healthcare staffing system.

FIG. 7 shows an example of an integrated platform for the smart healthcare staffing system.

FIG. 8 shows an example of a workflow managed by the smart healthcare staffing system.

### DETAILED DESCRIPTION

The presently disclosed subject matter provides a smart healthcare staffing system for hospitals. One embodiment of the current invention is a marketplace for on-demand nursing workforce. The staffing system may be a platform that connects together healthcare providers and per diem shift nurses (PRNs), where they may liaise directly, without a broker, resulting in just-in-time fulfillment of shifts. Through disintermediation and automation, the model may result in efficiencies (eliminating calls, paper and fax) and cost savings for providers who need temporary qualified nursing staff based on fluctuating demand, thus helping achieve patient satisfaction and quality of care. Nurses have flexibility and visibility into shift requirements, and better pay rates than going through a staffing agency.

The staffing system is a transparent, efficient, and streamlined approach that revamps the healthcare contingent staffing model. The platform may bring together healthcare providers with shifts that need to be filled, and nurses who may be able to fulfill that shift. The system allows healthcare professionals to choose when they want to work, where they want to work, and for who they want to work, based on location, schedule, rate, among other criteria.

The platform may match nurse profiles against the job requirements, including experience, licensure, area of specialty, competence, training, and any JCAHO/TJC (Joint Commission) requirements for the job. Matched nurses may be notified via a mobile application and can choose a posted job to accept a shift. Nurses may use a web-based portal to maintain their profiles, credentials, documentation, or any other information deemed pertinent. The platform may provide them the necessary tools to keep their licenses current.

Healthcare providers can interact with shift nurses via the healthcare provider application or the healthcare provider portal, to screen candidate licensure and competencies, before authorizing a shift. The platform enables easy job posting, job management, and review on completion.

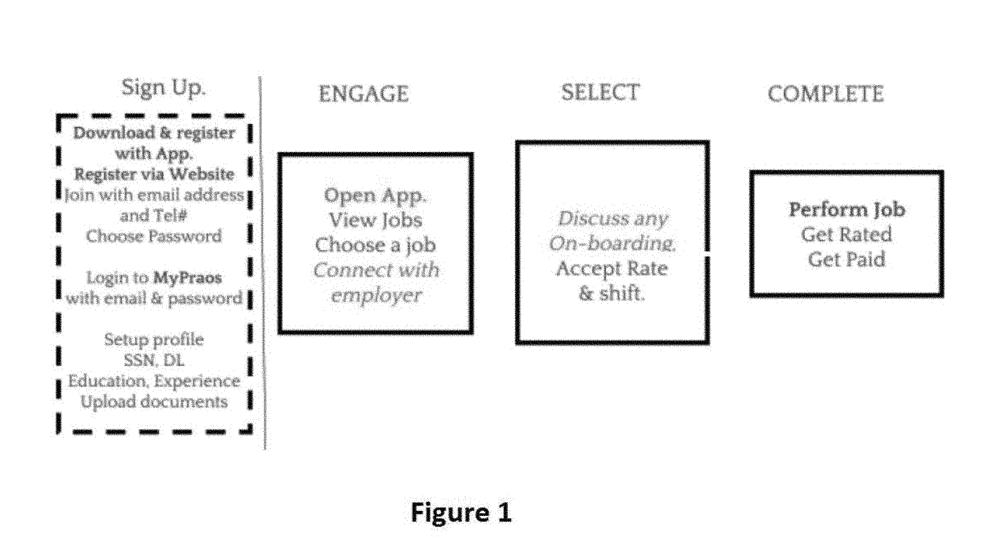

One example of field user work flow of the present invention is shown in FIG. 1. The initial step may be signing up to the portal. The step of signing up may include downloading the application and registering via website. Further, the user may set up an individual profile, including education credentials, pertinent experience, and accompanying documents. Within the signup step, the system may perform background checks and may contact the user for further screening and tests. Once the additional steps are completed, the user application may be approved.

Next, the user may open the mobile application, view and select jobs, and connect with an employer. Accordingly, the user has a degree of flexibility to choose a job as aligned with the user's needs based on the job supply. The platform may place the user in a direct contact with an employer once the selection is made. The selection process may include a discussion regarding on-boarding conditions with the hospital, which may further be facilitated by online on-boarding tools and courses. Once the agreement has been reached, and the rate is accepted, the completion notification may be sent to the employer. Subsequently, the user may check-in and the shift may commence. The shift may be governed by a work contract between the user (e.g., healthcare professional) and the hospital. Upon the shift being completed, the user may check-out, get paid and get rated by the facility management. The rating may be shared with the employee and recorded in a system database for review by future employers.

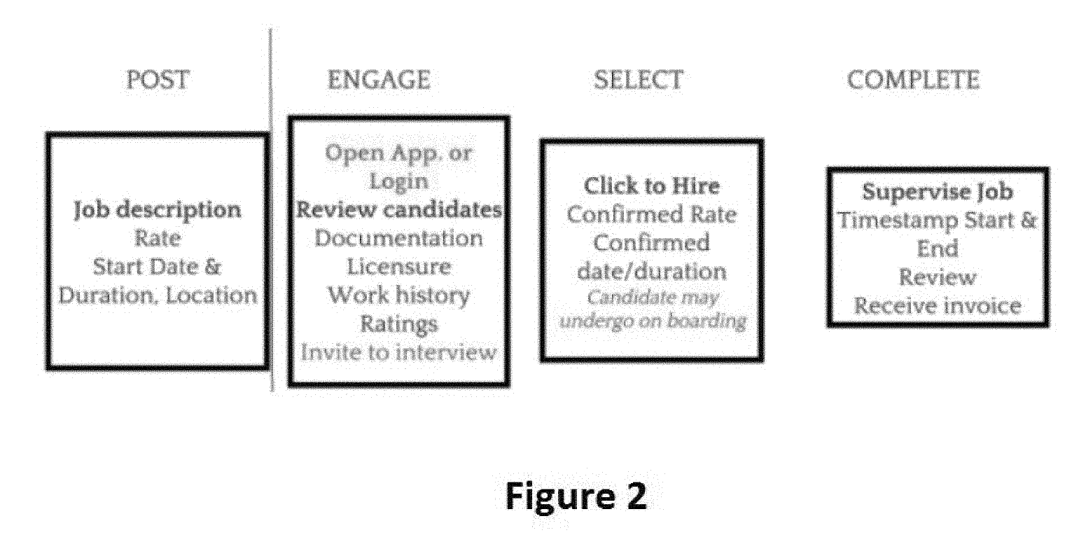

FIG. 2 presents an example of employer work flow of the present invention. An employer may use the system to post a job opening. The job post may include the description, the pay rate, the start date, the total required job duration and the location of the facility. The job opening may be posted via the mobile application or via the online portal. The platform may pre-screen the candidates and the applicants that match the job requirements may be instantly connected with the employer, i.e., a hospital.

The next step may be engagement between the employer and the hired medical professional. Upon opening the mobile application and logging-in, the employer may review the documentation submitted by the employee, such as work history, licensure, prior ratings entered in the system or any other documents deemed necessary. Moreover, the employer may set up an interview with the prospective employee. The engagement step may allow notifications to be sent to the hospital, or provide an express screen for negotiation. Further, once the click-to-hire option is selected, the pay rate and the employment period is confirmed, at which moment in time, the employee may begin boarding. Meanwhile, an employment contract may be created and executed.

During the employment period, the system allows the hospital to supervise the job performed by the employee. In addition to a start and end timestamp, the hospital management may enter a performance review of the employee, by providing feedback and rating. The rating system may promote top performers in comparison with the mediocre and unsatisfactory ones, and may allow future employers to make educated selection in terms of quality of work. Lastly, the system may send an invoice to the hospital and facilitate the payment to the employee that has completed the shift.

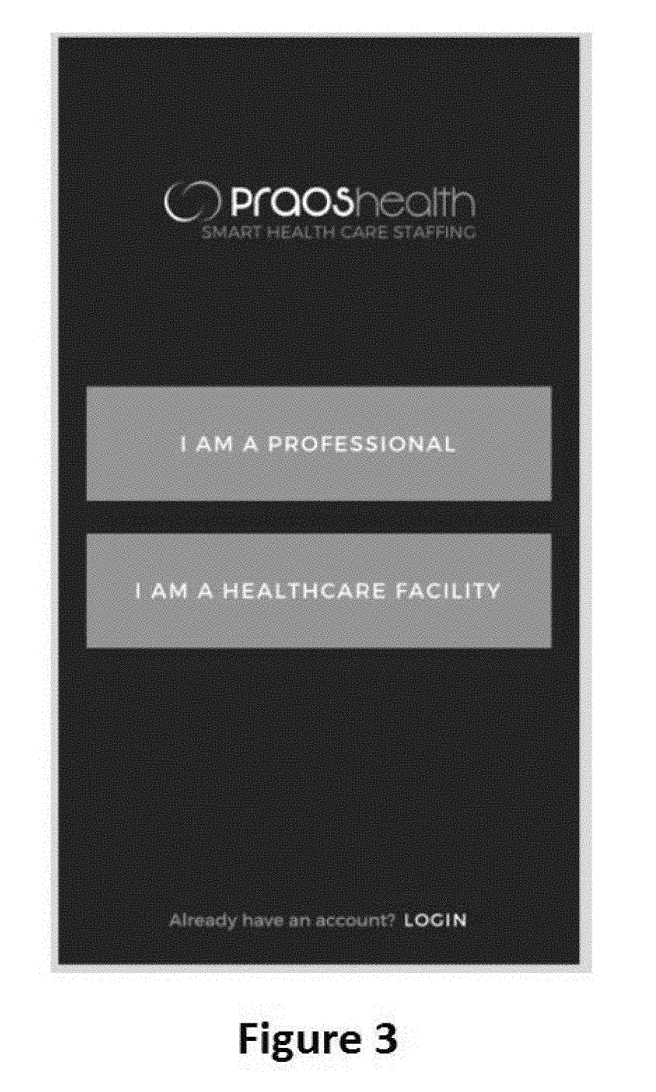

In terms of the sign up requirements, users may sign up for the service via a mobile application or via a website. The fields required for sign up may be: first and last name, email address, telephone number and a password. The users may download the mobile application from a variety of application stores currently available on the market. The users can scan QR codes on brochures or flyers or follow links from email campaigns or shared social media campaigns. Once the application is downloaded, the users can sign up with: email address, or a Facebook or LinkedIn profile, for example, as shown in FIG. 3. The mobile application may have Sign Up and Log In options for initial onboarding.

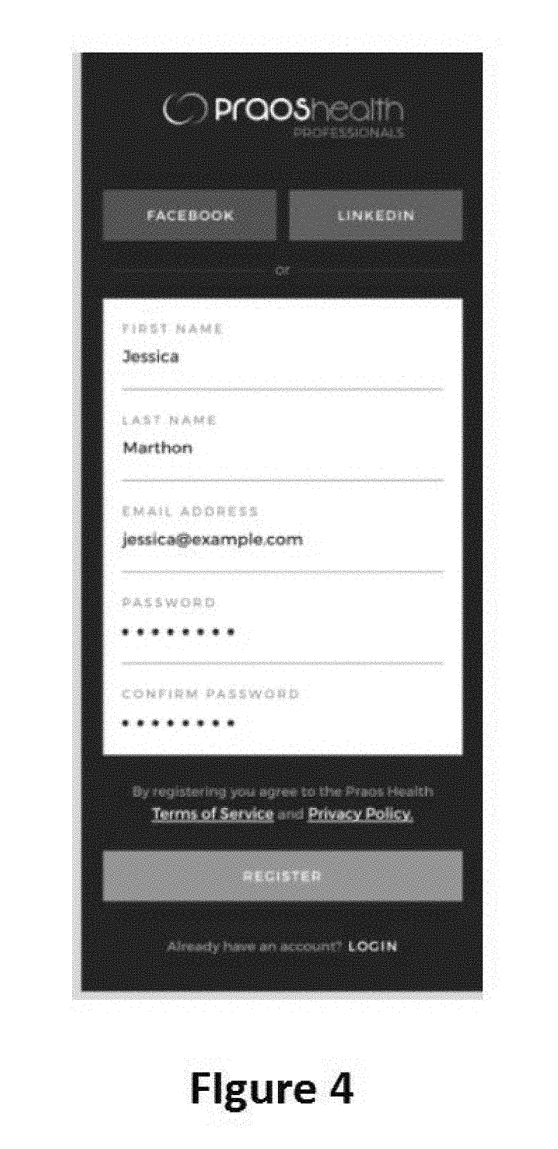

As exemplified in FIG. 4, an account may be created with the following fields: first name, last name, email address, telephone number, password, or any other information deemed suitable. The mobile application may incorporate language for agreement to privacy policy and terms of service for signup. The mobile application may display a progress dashboard until the application is completed with a prompt to complete application via the online portal. In the alternative, a user may sign up via the website by entering substantially the same information as in the mobile application. The website portal may be the central data and service repository for the users which includes account information, professional data and certifications, document storage, preferences, promotions, educational tools, job history, analytics and other information considered appropriate.

The website portal may include a home page, which may be the landing page of the portal. The landing page may showcase employers, continuing education training tools, including chosen onboarding training modules for preferred employers, relevant articles and links to health publications. There may be a central space dedicated to following active job notifications and discussions. With respect to job management in window, which may be available only for approved and active users, jobs past the current date may be marked inactive and archived for tracking in history system. Active jobs (current and future) can be selected to indicate availability and interest by the users by identifying status as—interested and available. Interested and available jobs may undergo screening or rate negotiation and job offer may be confirmed by employer thereby changing the status to—confirmed. Confirmed jobs can be accepted or declined by a user and the status may accordingly be set as—accepted or declined.

The application status in window may appear on signup and until application process is completed. This space may provide a welcome message on first login and show the status of application progress. The window may display an “Application under review” message when the application has been submitted. Links to privacy policy and terms of use may be included in the footer.

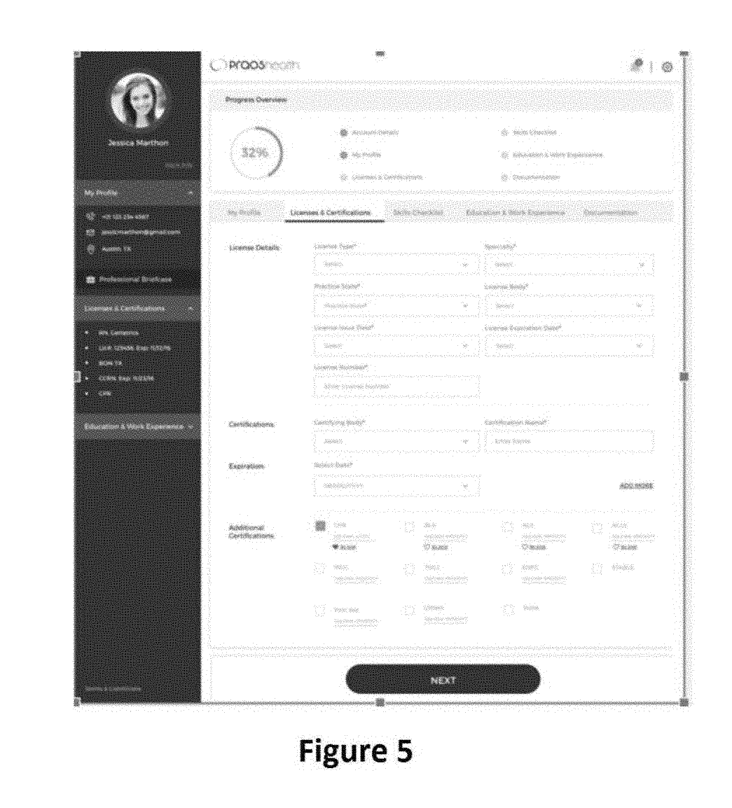

The portal may have links to the following functions: send message to the employer, view upcoming shifts, reset password, calendar view and synchronize, total number of hours worked (week, month, date range) and document export/share—share documents (secure link to employer). The header of the online portal may provide names, notification alerts (new messages), date, and log in/out status information. The portal may have several tabs and a sample layout is shown in FIG. 5.

A profile tab for applicant information may include name, address, contact email, contact telephone, emergency contact info (name, relationship, email, telephone), password, driver's license, social security number (encrypted to show only the last 4 digits), W9 Status, drug test, TB Test, vaccinations, gender, photo or any other information deemed appropriate. Private information in the profile tab (address, contact details, DL, SSN, etc.) may not be shared with unauthorized personnel or be available for public view.

A section regarding licenses and certifications may contain all the information pertaining to the license and certifications in addition to the body which grants and governs it. Data may be displayed as dropdowns for selection. A logical mapping to certifying bodies and associated licenses or certifications may be required. The skills checklist, required by hospital hiring managers, may be an important tool used to help match a user to an ideal assignment. Skills checklists often incorporate the needed certifications for the jobs required.

With respect to primary specialty, certifications and bodies may be displayed to the user based on their license: registered nurse (RN), licensed vocational nurse (LVN), nurse practitioner (NP), and clinical nurse specialist (CNS) and their corresponding practice area. For a nurse practitioner, for example, the combinations may be provided in the table below.

NN NEONATAL NURSE PRACTITIONER
NW WOMEN'S HEALTH NURSE PRACTITIONER
NP PEDIATRIC NURSE PRACTITIONER
NF FAMILY NURSE PRACTITIONER NP
NS SCHOOL NURSE PRACTITIONER NP
NG GERONTOLOGICAL NURSE PRACTITIONER
NA ADULT NURSE PRACTITIONER NP
NC ACUTE CARE ADULT NURSE PRACTITIONER
NE EMERGENCY NURSE PRACTITIONER
NR PERINATAL NURSE
NY PSYCHIATRIC/MENTAL HEALTH
NB ACUTE CARE PEDIATRIC

Further, for a clinical nurse specialist, the following table may apply:

CH: Community Health
CM: Maternal Child Health
CY: Psychiatric/Mental Health
CG: Gerontological Nursing
CS: Medical-Surgical Nursing
CP: Pediatrics
CN: Neonatal Nursing
CO: Oncology
CA: Adult Nursing
CW: Women's Health
CE: Emergency
CU: Adult/Gerontology Nursing

In addition, below is a table showing primary specialty selection for a registered nurse which may also apply to a licensed vocational/practical nurse:

A = COMMUNITY/PUBLIC HEALTH
B = GENERAL PRACTICE
C = GERIATRICS
D = OBSTETRICS/GYNECOLOGY
E = MEDICAL/SURGICAL
F = PEDIATRICS
G = PSYCHIATRIC/MENTAL HEALTH/SUBSTANCE ABUSE
H = ANESTHESIA
I = EMERGENCY CARE
J = HOME HEALTH
K = INTENSIVE/CRITICAL CARE
L = NEONATOLOGY
M = ONCOLOGY
O = REHABILITATION
P = OCCUPATIONAL/ENVIRONMENTAL HEALTH *Added
Sep. 4, 2001
Q = ANY OTHER PRACTICE AREA NOT LISTED ABOVE

In terms of additional certifications, the skills checklist section may provide a list of self-assessment forms. These skills checklists may give an overview of nursing experience and proficiencies based on the type of nurse. Providing such information is in line with TJC (Joint Commission) requirements for acute care hospitals. Many urgent care and home health agencies will require the candidate to have a completed form on file. The appropriate checklist may be selected from a dropdown and the completed data may then be stored against the account profile of the user. During the transaction negotiation process, healthcare providers may need to view to the checklist and download as part of their files, for audit purposes. The users may also download completed forms (stored against their profile) and/or share with employers via secure link their profile details/documents. Each of these skill checklists may be a unique self-review/evaluation form and a sample link may be provided to extract data fields for each form.

The system may provide education information of the candidate to the hospital. For example, for nursing professionals, available options from a dropdown menu may be diploma in nursing, associate degree in nursing, baccalaureate in nursing, master of nursing, doctorate in nursing, VN/PN program or any level of education acceptable for the job opening. In addition, a dropdown menu may be available to select a desired number of years of experience of the candidate, which may be accompanied by an uploaded resume and list of references.

The platform may include a hospital unit orientation and onboarding system, which may contain a computer skills dropdown menu. A resume building function may be incorporated to format the data into a standard resume format, which can be emailed to employers. Employers may also be able to access the resume, references, and documentation section directly via the portal for express checks and screening for those users who have indicated an interest in a job posting only.

Further, a documentation tab may be added for verification that the hired professional is current to be able to accept a shift job. The relevant documents may include a driver's license—stored in profile, liability insurance, driver's insurance, or any other documents required by the hospital. The status of these checks may be visible to the user. Any document and its content may be stored against the user account but not visible or accessible to the user. Documents may be uploaded to the user account once applicant background checks have been completed by a background checking company. In addition, documents can be viewed and managed by the platform support staff and may be released to the user under special request.

A laboratory and screening results database may include drug screening tests, lab work for TB tests, and any other specific health related screens. In terms of data security, this area may be password protected by default. Data may need to be encrypted on rest and during transmission. An upload feature may be available to upload documents (insurance, certifications, driver's license etc.) into the documentation area from a computer by the user.

The mobile application may be able to accept photos of documents (e.g., license certificates) and securely auto-upload it to this area, e.g., depositing checks from the mobile application. A user may be able to share any stored lab documents via the platform (one time secure share link) for each ‘share’ request with a potential employer. The access to this sensitive information may be restricted for any platform administrator.

In order to use the staffing systems, users may need to complete the application via the portal, upload necessary documentation, and set preferences. Online self-help video and tutorials may be provided. The account status during application may be any of the following states: Initiated: at user sign up; Pending: after 1 week of signup and application not submitted; Submitted: when applicant completes all the mandatory information, where the information is automatically submitted to the helpdesk; Verification: when verification has begun; Verified: when the verification is completed successfully and the candidate becomes available in the pool (account status changes to Active); Rejected: if there were unresolvable issues with background checks or licensing in which case the applicant is notified; De-registered: data is sanitized after 60 days. Applicants can be manually put back on ‘Pending’ to renew licenses. While expired licenses may not be acceptable, expired certifications may be acceptable for completing the application as nurses can still work with their basic RN license, for example.

Many acute care hospitals have a requirement for nursing staff to undergo orientation/onboarding using their specific training modules. Based on the home location of the user, the website home page may display featured hospitals and recommended onboarding and orientation tools that may need to be completed in order to be placed at these facilities. Training may be online and a certificate or confirmation may be provided on successfully completing the training. Results of training completion may be stored against the profile of the user. Details of completed training modules may appear in this section, along with the digital certificate of completion. If onboarding is a requirement for the job, only appropriately trained nurses may receive the job notification.

Maintaining licensure normally requires nurses to follow a certain number of hours of continued education as stipulated by the State Board of Nursing. Continued education (CE) courses are often taken online and credits are given for attending certain seminars or industry related conferences. A section in the staffing system may be allocated to maintain all documentation pertaining to completing necessary CE courses and may be readily available to submit as proof for licensure renewal. The platform may also provide CE courses through certified course partners and links may be provided via the portal.

In terms of account setup, ensuring timely and accurate completion of the application sections may be achieved with a dashboard that may allow monitoring progress of essential functions. The dashboard may be displayed on the website homepage. Automated email and mobile alerts may be provided over the course of the application lifecycle.

An initial welcome email may be sent with a user name, a password, and links to tutorials and videos to complete the application, as soon as a user registers via the website or the mobile application. At the outset, the account may be created. A progress email for applications in the initiated state with a dashboard snapshot may be sent every other day (for 7 days) to encourage users to complete the application in a timely manner. Specific instructions and tutorials pertaining to incomplete sections may be included in the emails.

After 7 days, unsubmitted applications may be set to ‘Pending.’ Reminder emails may be sent on day 15, day 30, and day 45 (de-registration warning email). On day 60, a de-registration email may inform the user that the registration has been aborted. In this case, users may not be able to login any longer and account may be deleted. Applicant registration information may be cleaned out after 60 days of non-completion of the application and an email may be sent to the user for de-registration. The account may be deleted and an error message may be sent to the mobile application in case users are trying to login. However, field users may sign up again with the same email—but the process may need to start over.

Completed applications which are automatically submitted may be queued for background and credential screening with the platform support desk. The support personnel may verify all the information and initiate the background check and licensure check process. A call to the applicant may be required as part of the background check. All applicants may go through a screening process (background check, drug screening, licensure verification, etc.) as described above. Job applicants can be contacted for further information, including initiating lab test result requests, by a support person. Any lab or health records of the users may be stored in a HIPAA compliant fashion and released to prospective employers directly by the user. Once the user is vetted successfully, the application process may be deemed complete and the user may be placed in the ‘aggregate’ work pool.

A user account may have three states—active, suspended and inactive. The active status may occur when a user is placed in the aggregate work pool after application is verified. Active accounts are live and receive job notifications, alerts, emails, promotions, and continuing education offers. The portal may be updated with work history logs and reviews. The account may be suspended for inactivity or non-compliance such as license expiration. Suspended accounts may not receive job notifications, or offers. Only certain alerts and reminder emails may be sent. Suspended accounts may be able to login to the portal to update any documents, profiles, etc. Viewing may be restricted to account information only. The homepage may display a default message and no active promotions or offers may be viewable. An inactive account may be closed resulting in no communication or activity authorized on the account. Unsuccessful login to the platform and mobile application may display an error message.

Accounts may be checked for license expiration and tagged for renewal reminders. Some of the categories included in monitoring and reminders may be nursing license (Priority), driver's license, certifications—as provided in the certifications tab, motor vehicle insurance, liability insurance, or any other categories deemed suitable. Reminder emails and mobile alerts may be sent periodically—starting 3 months prior to expiration date. The emails may be triggered based on checking the current expiration date (since license could be renewed in the interim). The emails may be triggered according to the following example: expiration month—2: generic reminder email to renew; expiration month—1: generic reminder email to renew; expiration month (start of month)—renewal reminder plus warning that they will not be able to receive job notifications; expiration month (14 days and 7 days prior to expiration date): renewal reminder.

Nursing license expiration may result in ineligibility to work as a nurse in their licensed state. Links to the state BON for renewal maybe provided. On expiration of the nursing license, the account may be suspended and a suspension email may be sent. For other types of expirations, a notice of expiration may be sent. Automatic licensure verification via API calls may be performed on a daily basis (batch) and relevant state systems may be checked for expiration, violations, disciplinary action etc.
Healthcare providers may sign up for the service via the platform website. Healthcare providers may fill out a form with their contact details and may be contacted by an account representative to create the account. As part of the account setup process, healthcare providers may need to provide a disbursement and billing number and/or a valid credit card for billing. In phase1, billing may be facilitated via credit card and pre-authorization of the healthcare provider credit card may be performed at sign-up. The staffing platform may provide invoicing to qualified healthcare providers on an as-needed basis.

Healthcare provider accounts may be set up on the platform and healthcare providers may be provided login credentials for the healthcare provider portal. Healthcare providers can also download the healthcare provider application to and communicate with candidates interested in posted jobs. The portal may enable healthcare providers to post jobs, review and screen candidates, share jobs, authorize shifts (contract), and timestamp shifts. Reports and analytics may be available via the portal in addition to billing and invoicing information. Once logged in, healthcare provider users may be able to see their logo on the portal pages (co-branding). Healthcare providers may be provided online training tools to educate them on the platform features. Training videos and customer care may be available to help assist job posting and management of users within the organization.

In large facilities, such as acute care hospitals, a system administrator may sign up for the staffing service and there may be multiple individuals from different departments posting jobs belonging to different units, such as, for example, a charge nurse in cardiology posting for a CVICU nurse and an HR person posting for a NICU (neonatal ICU nurse) for a different unit. Thus, a hierarchy of users, roles, and privileges belonging to departments within an organization may be available.

The platform may support a tiered structure for healthcare providers. Some of the user roles may be: charge nurse (job responsibilities include posting, viewing, editing, and managing their own jobs; viewing across department or group); nurse director/group manager (posting, viewing, editing and managing for all group users); admin (job description involves all of the above in addition to user setup).

A healthcare provider portal may be the healthcare provider view to the platform. The healthcare provider portal may be the repository for all facility and unit profiles, job descriptions, rates, job authorization and management, job history, reports, analytics, billing, payments, and invoicing. All job information may be detailed under current jobs, jobs in progress, unfulfilled jobs, fulfilled jobs, deleted jobs, and overall job history.

The portal's administrative function may allow creation and management of departments, closed user groups, and end users. Jobs can also be prioritized for certain groups in a certain order so that notifications sent to the priority group may take precedence over other groups. Rules can be set on time limits, or states (unfilled by a certain deadline) before the next group is notified. The facility and unit profile tab may showcase the healthcare provider facility description, such as hospital, home health, description, link to the website, etc. Content may be uploaded directly by the healthcare provider.

Regarding tools and templates available to create a job, once a job is created, a user may be able to: edit a job, save a job, post a job (post a saved job), edit a posted job and re-post a job, copy a job, delete a job, or share a job, for example. Jobs can be created using the Freeform tab, imported from a file, via API into existing systems. Job headers may use the standard template provided below. The Freeform tab may provide the flexibility to cut and paste job descriptions from existing documents.

An example of a job description may be the following:
The Clinical Nurse II safely practices with the hospital's professional practice model and demonstrates the hospital's core values, which reflect its mission. The Clinical Nurse II provides direct care to patients who may range in age from infancy to the elderly and demonstrates competency within the domains of clinical judgment and decision-making, nurse-patient/family relationship, clinical leadership, and clinical scholarship. The CN II maintains a competent level of knowledge and skill while providing direct care. The nurse demonstrates the knowledge and skills necessary to provide individualized care based on physical, psychosocial, cultural, educational, safety, and age-appropriate considerations of assigned patients. The nurse complies with all regulations and standards of regulatory and accrediting bodies.

In terms of schedule, the scheduling information may provide start date and time. Scheduling can be done up to a month in advance. Future phases may include booking a shift for nurses in the favorites list, within a closed user group, or float pool. The platform may provide an ability to synchronize with the calendar of the user, shared third party, etc. For audit trail, chronological listing of all created, edited, deleted, authorized, cancelled, posted, re-posted jobs may be included.

Billing, payment and invoicing may be executed by credit card, or any other payment means deemed appropriate. The payment tab may have the healthcare provider credit card information. The card number may be encrypted and only last four digits can be viewed. The healthcare provider can update credit card information and a pre-authorization may be performed on the new card before it is stored successfully. Credit card name and expiration fields may be managed by the healthcare provider. Expired credit cards may have an automatic email triggered to the billing contact of the healthcare provider—flagging expiration.

Once the shift is approved, the credit card on file may be pre-authorized for the invoice amount. The invoice amount may be calculated by multiplying shift hours with the billable rate, in addition to the fees for using the platform. If a shift is authorized prior to 24 or more hours in advance, then pre-authorization may occur 24 hours before the shift begins. A pre-authorization may be completed at the earliest among: the time of shift authorization, the day the shift is scheduled, or six hours before the shift commencement. The card may be charged for the invoice amount when the shift is complete and time stamped.

Failed transactions may immediately be alerted to the accounting/finance department and the healthcare provider account may be suspended. Suspended healthcare providers may not have the ability to post new job descriptions and the system may cancel scheduled jobs until payment is guaranteed. Conversely, pre-authorized charges may be removed when the healthcare provider cancels a scheduled job. A minimum notice period of 24 hours may be required for job cancellations. Jobs posted within a 24 hour window and accepted by a user may incur a fee.

Invoices on completed jobs with the invoiced amounts, date, department, job ID, username, rate, and total hours, may be available to the healthcare provider. Invoices may be paid on a per job basis. Reports on total invoiced amounts and details of invoices may be generated on a daily, weekly, and monthly basis, based on the healthcare provider preferences. Invoices may be emailed as PDFs to the billing contact and a copy may be stored in the healthcare provider portal.

The analytical reports may include information such as: total jobs, jobs completed (daily, weekly, monthly), job ID, number of hours, rate, department, average rate of fulfilled jobs, unfulfilled jobs (daily, weekly, monthly), average rate for unfulfilled jobs, total invoiced amounts, productivity savings metrics, average time to fulfill, quality metrics, number of unique RNs staffed, number converted to float pool or permanent hires and other information considered relevant for the monitoring of performance and fulfillment.

In order to find a match between a job opening and a suitable candidate, the algorithm may look for certain keywords in the job description and match them to the user information as entered in the database. The matching process may use a set of criteria which includes: a hierarchy of rules based on certification and skills, job title, job category, licensure, or any other criteria considered adequate.

The job location may be matched to a state that the user is licensed to practice. In other words, if there is a location match, then the job category may be matched. Once there is a licensure state match, the job category may need to be matched next. There may be certain mandatory matching requirements on the following job categories and certifications, including the words pertaining to those categories: RN—registered nurse (RN, nurse, PRN, per diem nurse, per diem RN); LVN—Licensed vocational nurse (LPN—LVN Licensed practical nurse, LPN, LVN, vocational nurse, practical nurse, nurse assistant, per diem LVN, per diem LPN, etc.); NP-Nurse Practitioner (Nurse practitioner, NP, certified NP, certified Nurse practitioner, per diem NP, CNP, etc.); CNS-Clinical Nurse specialist (Nurse specialist, clinical nurse specialist, CNS, etc.); PA-physician assistant (PA, physician assistant); CRNA-Nurse Anesthetist (NA, nurse anesthetist, CRNA, certified nurse anesthetist); CNW—Nurse midwife (Nurse Midwife, RN midwife, CNW).

The license expiration date may be checked for validating the availability of the candidate. Notifications with expired licenses may be disallowed. When a certification is specified, its issuance may be mandatory. The certification requirements may be matched to fields in the users' additional certification fields. Specialty certifications and onboarding training may be certifications for specialty courses awarded by the various boards. In most cases, short term shift nurses may not be required to be board certified. However, when the healthcare provider requires the certification as part of the job, an exact match may be required to the corresponding certification of the user. The certification expiration date may be checked for validity.

If the healthcare provider specifies that onboarding/orientation training is a requirement, then only appropriately trained/oriented nurses may be matched. The orientation list of the user may be checked to ensure that the hospital is on that list. Nurses with similar background can get an alert of the job and a reminder/message that online orientation training is recommended to gain jobs from the healthcare provider.

Tag words may be incorporated in the staffing platform to match keywords from job description to skills checklist and resume strings, such as Cardiology, ICU, Trauma, Stroke, Pediatric, Neonatal, Rehab, Psych, Med Surge, Surgical, CVICU, Cardiac, CV, ICU, BCLS, ACLS, PICU, Pediatric ICU, critical care, ICU, CCRN-Pediatric, etc.

Once matching profiles with valid licenses are found, job notifications may be sent to the user. By default, all notifications of matched jobs may be made available on the portal, similar to email messages and can be viewed, accepted, or deleted, for example. Matched jobs may also be notified via email. The portal may be capable of setting up different search filter criteria, which can also be set from the mobile application, as will be described below. The platform notifications can be searched by location, date, healthcare provider, rate, and by any other criterion considered appropriate.

There may be two versions of the mobile application, one created for the candidate and another one for the hospital. The user mobile application can control the settings for receiving notifications based on user selected options. The user can turn OFF and turn ON notifications, the default option being ON in one instance. The mobile application may provide the ability to have both a ‘home location’ and a ‘current location’ search. The user can initiate a ‘search for jobs near me’ function. The GPS location of the user may be used to locate matching jobs in the location vicinity of 100 miles radius. The user can add specific health care providers to the ‘favorites’ list and opt to receive notifications only from favorites.

The user mobile application may be able to perform multiple functions upon logging in. One example is viewing account details (name, email, password) and profile information (job category, license type and expiry, certifications and expiry dates). Another function may be job menu, which may present a list of job notifications (current and future) for a search by date, location, rate, etc. The job menu may contain a default listing in chronological order or expired jobs that are purged from the list. Further, the favorites function may list healthcare providers marked as favorite, and the Myjobs function may present accepted/upcoming jobs, or past jobs. In addition, the MyIncome function may state total hours worked and the consequently earned income. The job actions function may pertain to valid and currently unaccepted jobs, and may allow selecting an interested job, accepting the job, scheduling to calendar, cancelling an accepted/scheduled job, messaging employer, or sharing documents with employer, for example. The job alerts function may be used for new jobs, reminders (e.g., one day prior and two hours prior), license expirations, login/system messages, payments, cancellations etc. Other functions such as driving directions, timestamp (job logging), job review (employer rating), and error messaging (when no matching jobs are found, for example), may be incorporated in the staffing platform.

The healthcare provider mobile application may allow a view account details option (name, email, password), the post-a-job option (by shift, from favorites, to a particular group), the job management menu (list of current future jobs by date, job title), default listing in a chronological order, list of posted jobs, list of candidate responses, rejected jobs (either candidate or healthcare provider rejects), authorized jobs, jobs in progress, etc. The mobile application may further provide a job actions tab showing valid, interested, or accepted jobs, or jobs in progress, or may also facilitate a view of documents on interested job and message the user.

The hospital mobile application may permit rejecting an interested job (from candidate), authorizing job, scheduling authorized jobs to calendar, canceling a scheduled or authorized job, showing jobs in progress and approving timestamps, for example. Appropriate notifications may be sent on interested jobs and time stamp requests and the consequent action may be performed such as: approve timestamp, start job, end job, etc. Lastly, the mobile platform may provide a review tab, where the user may be rated upon the job completion.

One example of the present invention allows hiring aggregation, which may result from availability of verified candidates with a current valid license for placement. Candidates with expired licenses may not be available in the aggregate pool for placement until their license is renewed and verified.

The healthcare provider may be able to view a candidate's stored profile and data in the staffing platform. The profile may be screened based on the health care providers' criteria such as blocking a list, or a current employee, of eliminating a candidate with invalid license and certification. The healthcare provider may also communicate with the candidate regarding any specific questions on experience or the provider's onboarding requirements. Once requirements are met, the health care provider may be able to authorize the job/shift. This process may be referred to as the transaction negotiation.

Any rate or date or location may change results in an edit and re-post of the job. The ability to re-post to a single candidate or to the marketplace may be required. Based on the health care provider's authorization, the work contract may be generated for the number of hours, rate, on the set date and at the location, as set out in the job description.

Future phases may include billing on a daily or weekly or monthly basis, with department roll up, subject to certain minimum account balances being maintained. Payments may be made to the employee's bank account within a predetermined period of time, such as, for example, 1-3 business days of job completion. Bank account information may be captured for each employee and stored in the system for payments. Payment effected alerts may be sent to the mobile application and the portal and details of payment may be recorded against the employee's account. Details can be viewed on the portal.

A dashboard may be used by the platform administering company to manage the business. The dashboard may provide insight into healthcare provider account activities, total number of jobs posted on the platform, per healthcare provider, by day, by location etc. Further, support and maintenance issues may be resolved and system alerts may be provided on the platform. An insight on candidate distribution on the locale level may be available, as well as access to individual account fields for maintaining background checks, notes, manual status updates, account management etc. The dashboard may be used to post or manage a job manually on behalf of the provider or to moderate a review and intervene based on investigation of a complaint.

The rating of nurses may be done via the mobile application and the healthcare provider portal as soon as the task ends and the job is time stamped by the supervisor at the healthcare provider. The rating may range from one-star to five-star from the lowest to the highest. Ratings may not be published instantaneously. Instead, they may be moderated by the platform where complaints, violations, no-shows are investigated. Ratings may be attributed to the nurse's profile and can be seen by healthcare providers whose job postings have been accepted by the nurse, for example.

Nurses may be rated based on numerous criteria, such as punctuality, professionalism, team work, skills and competency, attitude, cancelation metrics or any other criterion relevant to the performance. The cancellation meter may be system computed and may show the total number of shifts accepted, total cancelled, and the percentage of cancellation. A graphic icon may be presented to illustrate the percentage.

Cancellation metrics may be shown on the profile along with rating. The rating of hospitals may be done by the nurse on completion of the job. Hospitals may be rated based on ease of getting oriented, team work, environment, supervisor, employer job meter or any other metrics deemed suitable. The rating may be system generated and may show the number of jobs posted, number of cancelations, percentage of cancelations, number of job authorizations, and percentage of authorizations in graphical format.

With respect to job fulfillment, standard on-demand mobile tracking features may be available. Jobs may be time stamped by the employee and accepted or endorsed by the healthcare provider. A start time stamp and an end time stamp may be at the location of the work contract.

Several vendors may be hired to provide background screening services to the staffing platform, such as Backgroundchecks.com, Chekr.com, sterling talent solutions, Hireright.com, to name a few. Some vendors may provide APIs and hooks may be in place to develop API calls and automate background check requests and upload results on the staffing platform. Required background checks may be: multi-jurisdictional criminal database search, driving record report and additional checks may be available paid by the employee or the healthcare provider as-needed, such as drug screening or TB tests. The platform website may be used to disclose background check to candidates via a link to the website, when the user approves the background check via a tick box.

The staffing platform may verify the licensure status of all nurses seeking employment. Many state boards of nursing have gone paperless and no longer issue a wallet size license card. An online verification system may be used for licensure data obtained directly from the licensure systems of boards of nursing through frequent database updates.

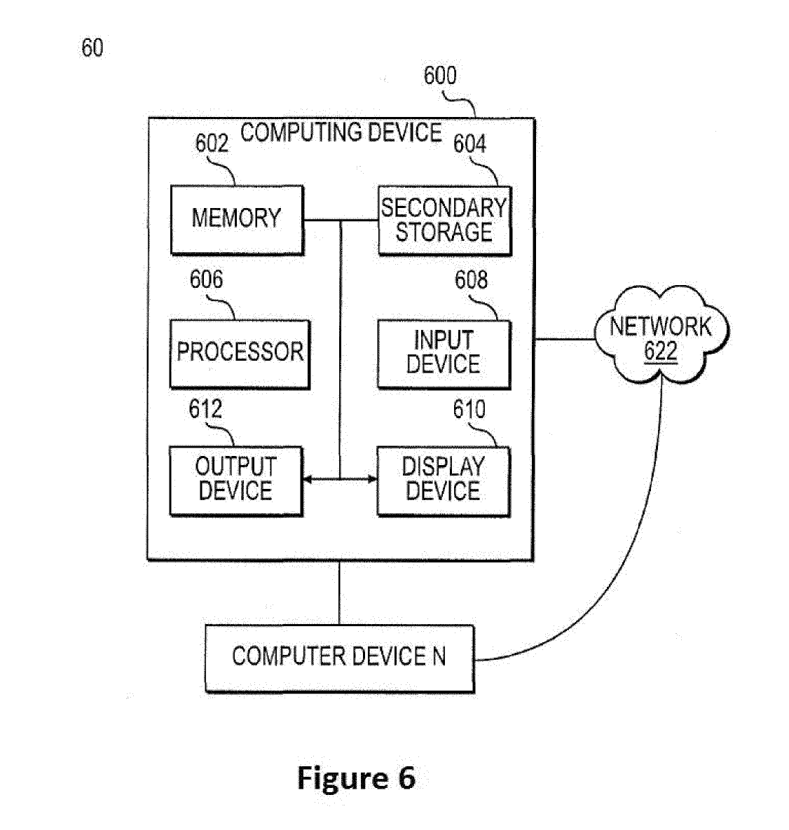

The smart healthcare staffing system for hospitals may be performed on a computing device as shown in FIG. 6. A block diagram of FIG. 6 illustrates a system 60 that includes one or more networked computing devices or systems 600. System 60 may include a server computing device 600 to make the connections and/or run the processing on multiple client or otherwise networked computing devices 600. Computing system 600, including client-servers combining multiple computer systems, or other computer systems similarly configured, may include and execute one or more subsystem components to perform functions described herein, including steps of methods and processes described above.

Computer system 600 may connect with network 622, e.g., Internet, or other network, to receive inquires, obtain data, and transmit information and incentives as described above. Computer system 600 typically includes a memory 602, a secondary storage device 604, and a processor 606. Computer system 600 may also include a plurality of processors 606 and be configured as a plurality of, e.g., bladed servers, or other known server configurations. Computer system 600 may also include an input device 608, a display device 610, and an output device 612. Memory 602 may include RAM or similar types of memory, and it may store one or more applications for execution by processor 606.

Secondary storage device 604 may include a hard disk drive, CD-ROM drive, or other types of non-volatile data storage. Processor 606 executes the application(s), such as subsystem components, which are stored in memory 602 or secondary storage 604 or received from the Internet or other network 622. The processing by processor 606 may be implemented in software, such as software modules, for execution by computers or other machines. These applications preferably include instructions executable to perform the system and subsystem component (or application) functions and methods described above and illustrated in the herein. The applications preferably provide graphical user interfaces (GUIs) through which users may view and interact with subsystem components (or application in a mobile device).

Computer system 600 may store one or more database structures in secondary storage 604, for example, for storing and maintaining databases and other information necessary to perform the above-described methods. Alternatively, such databases may be in storage devices separate from subsystem components. Also, as noted, processor 606 may execute one or more software applications in order to provide the functions described in this specification, specifically to execute and perform the steps and functions in the methods described above. Such methods and the processing may be implemented in software, such as software modules, for execution by computers or other machines. The GUIs may be formatted, for example, as web pages in HyperText Markup Language (HTML), Extensible Markup Language (XML) or in any other suitable form for presentation on a display device depending upon applications used by users to interact with the system (or application).

Input device 608 may include any device for entering information into computer system 600, such as a touch-screen, keyboard, mouse, cursor-control device, touch-screen, microphone, digital camera, video recorder or camcorder. The input device 608 may be used to enter information into GUIs during performance of the methods described above. Display device 610 may include any type of device for presenting visual information such as, for example, a computer monitor or flat-screen display (or mobile device screen). The display device 610 may display the GUIs and/or output from sub-system components (or application). Output device 612 may include any type of device for presenting a hard copy of information, such as a printer, and other types of output devices include speakers or any device for providing information in audio form.

Examples of computer system 600 include dedicated server computers, such as bladed servers, personal computers, laptop computers, notebook computers, palm top computers, network computers, smart phones, mobile devices, or any processor-controlled device capable of executing a web browser or other type of application for interacting with the system.

Although only one computer system 600 is shown in detail, system and method embodiments described herein may use multiple computer system or servers as necessary or desired to support the users and may also use back-up or redundant servers to prevent network downtime in the event of a failure of a particular server. In addition, although computer system 600 is depicted with various components, one skilled in the art will appreciate that the server can contain additional or different components. In addition, although aspects of an implementation consistent with the above are described as being stored in memory, one skilled in the art will appreciate that these. aspects can also be stored on or read from other types of computer program products or computer-readable media, such as secondary storage devices, including hard disks, or CD-ROM; or other forms of RAM or ROM. The computer-readable media may include instructions for controlling a computer system, computer 600, to perform a particular method, such as methods described above.

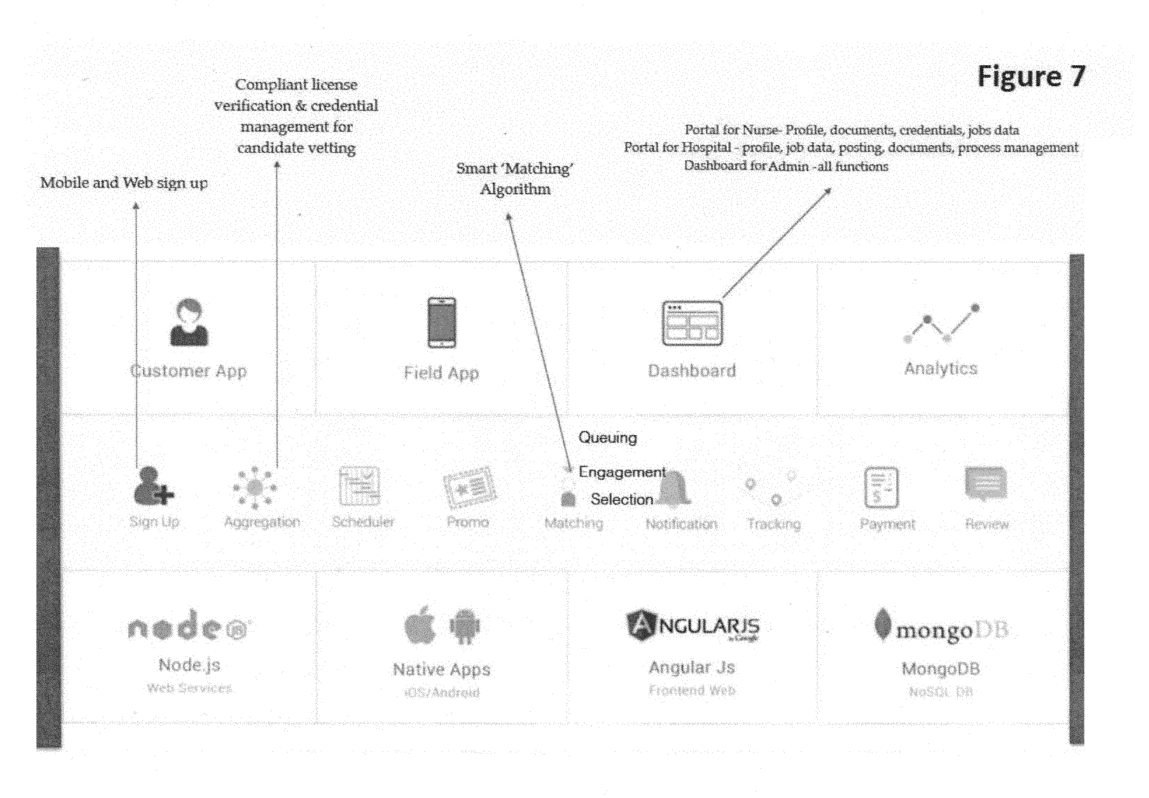

FIG. 7 depicts an example of an integrated platform for the smart healthcare staffing system. As described in detail above, the platform may include mobile and website portal signup and a vetting system for credentials and compliance. Further, the integrated platform may incorporate a matching algorithm for queuing, engagement and selection of candidates. A dashboard may be customized for nurses, hospitals, or system administrators. Once the job is confirmed by the hospital, i.e., when the candidate is chosen, the platform may automatically block the candidate's calendar, send them shift reminders and provide geo-tracking of the candidate to the job while enroute to the job.

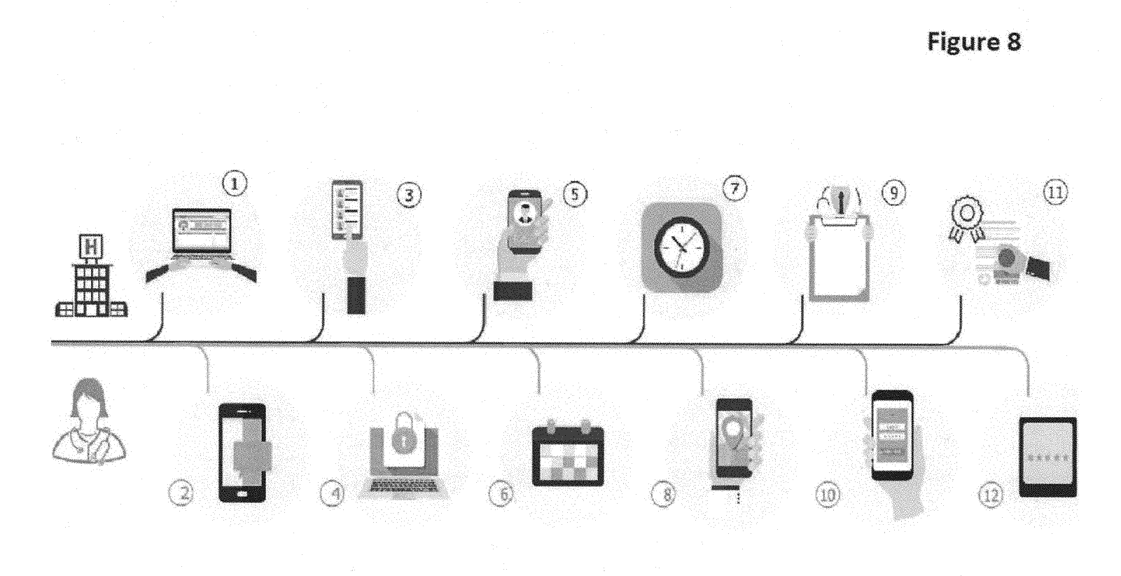

FIG. 8 shows an example of a workflow managed by the platform. In step (1), a hospital may use a template provided by the platform to post a job opening and specify individual requirements, such as rate and shift details. A candidate may, in step (2), receive an instant notification on a mobile application based on the candidate's credentials, qualifications and work preferences. The candidate may apply for a shift based on availability, facility profile, and rate, for example. The next step (3) may include screening of the candidates by viewing the complete profile with experience, credentials, certifications, skills, etc. Next, the candidate may share the requested documents with the hospital in step (4). In turn, the hospital management may make a hiring decision in step (5), assign a supervisor and specify orientation modules. In step (6), the calendar is scheduled for the selected candidate, who may prepare for the shift. The platform may further allow the hospital to stay updated on the hired staff's arrival time, as shown in step (7). Upon arrival, in step (8) the hired staff may arrive at the work location and care for patients. The hospital may further use the platform in step (9) to supervise the shift and provide a safe working environment. In step (10), the staff may use the mobile application, for example, to clock the shift. Step (11) allows the hospital to approve the timestamp and rate the nurse. Lastly, in step (12), the nurse payment may be facilitated and an option for rating the facility may be provided.

Although the various systems, modules, functions, or components of the present invention may be described separately, in implementation, they do not necessarily exist as separate elements. The various functions and capabilities disclosed herein may be performed by separate units or be combined into a single unit. Further, the division of work between the functional units can vary. Furthermore, the functional distinctions that are described herein may be integrated in various ways.

The foregoing description and examples have been set forth merely to illustrate the invention and are not intended to be limiting. Each of the disclosed aspects and embodiments of the present invention may be considered individually or in combination with other aspects, embodiments, and variations of the invention. Modifications of the disclosed embodiments incorporating the spirit and substance of the invention may occur to persons skilled in the art and such modifications are within the scope of the present invention.

What is claimed is:

1. A networked computerized system for integrating and processing staffing information, comprising:
a plurality of networked, standalone, programmed devices; and
a network that connects the networked, standalone, programmed devices;
wherein each of the plurality of networked, standalone, programmed devices includes:
a data storage subsystem component that stores information about at least one employer and at least one staffing candidate;
a data entry subsystem component that allows input of hiring requirements of the at least one employer and of credentials of the at least one candidate,
a data analytics component programmed to compare the hiring requirements with the credentials and to produce a match between the hiring requirements and the credentials, and
a workflow management component programmed to manage a professional engagement resulting from the produced match,
wherein the data analytics component that produces the match and the workflow management component that manages the professional engagement are integrated in the networked computerized system.

2. The system of claim 1, wherein the workflow management component is used for negotiating terms of employment of the at least one candidate.

3. The system of claim 1, wherein the workflow management component is used for compensation of the at least one candidate.

4. The system of claim 1, wherein the workflow management component is used for performance rating of at least one of the following: the at least one candidate and the at least one employer.

5. The system of claim 1, wherein the workflow management component is used for supervision by the at least one employer of job performance of the at least one candidate.

6. The system of claim 1, wherein the workflow management component provides performance review data to the data storage subsystem component.

7. The system of claim 1, wherein the staffing information contains at least one of the following: healthcare, education, security, drilling, disaster recovery and emergency information.

8. A method for integrating and processing staffing information, comprising:
storing information about at least one employer and at least one staffing candidate by using a data storage subsystem component of a networked computerized system,
inputting hiring requirements of the at least one employer and of credentials of the at least one candidate by using a data entry subsystem component of the networked computerized system,
comparing the hiring requirements with the credentials and producing a match between the hiring requirements and the credentials by using a data analytics component of the networked computerized system, and
managing a professional engagement resulting from the produced match by a workflow management component of the networked computerized system,
wherein the data analytics component that produces the match and the workflow management component that manages the professional engagement are integrated in the networked computerized system.

9. The method of claim 8, wherein the staffing information contains at least one of the following: healthcare, education, security, drilling, disaster recovery and emergency information.

10. The method of claim 8, wherein the workflow management component is used for negotiating terms of employment of the at least one candidate.

11. The method of claim 8, wherein the workflow management component is used for compensation of the at least one candidate.

12. The method of claim 8, wherein the workflow management component is used for performance rating of at least one of the following: the at least one candidate and the at least one employer.

13. The method of claim 8, wherein the workflow management component is used for supervision by the at least one employer of job performance of the at least one candidate.

14. The method of claim 8, wherein the workflow management component provides performance review data to the data storage subsystem component.
